(19) United States
(12) Patent Application Publication
(10) Pub. No.: US 2011/0160531 A1
(43) Pub. Date: Jun. 30, 2011

(54) NATURAL ORIFICE TRANSLUMENAL ENDOSCOPIC SURGERY (NOTES) DEVICE

(76) Inventors: Yun-Hen Liu, Guishan Shiang (TW); Ching-Yang Wu, Linkou Township (TW); Po-Jen Ko, Taipei City (TW)

(21) Appl. No.: 12/649,669

(22) Filed: Dec. 30, 2009

(51) Int. Cl. A61B 17/34; A61B 1/00

(52) U.S. Cl. 600/106; 606/185

(57) ABSTRACT

A natural orifice translumenal endoscopic surgery (NOTES) device includes a puncture needle, dilator sheaths and working sheaths. The puncture needle includes a puncture end and a protruding safety stud situated substantially at a middle section of the puncture needle. Each dilator sheath has an insert end and a position limit end, and each dilator is sheathed sequentially onto the puncture needle, and the position limit end is stopped by the safety stud of the puncture needle, such that the insert end will not exceed the puncture end and will form a safety interval from the puncture end. The working sheath is provided for inserting and retracting the dilator sheaths and the puncture needle to form a space required for a surgical operation after the dilator sheaths have expanded a natural orifice, so as to provide a safe and convenient use of NOTES.

### FIELD OF THE INVENTION

The present invention generally relates to a technical field of surgical device, in particular, to a natural orifice translumenal endoscopic surgery (NOTES) device.

### BACKGROUND OF THE INVENTION

At early stage, a surgical operation such as a thoracic surgery generally leaves a large wound and causes a serious postoperative pain to patients. As modern medicine and medical instruments have been advanced, minimal invasive surgery (MIS) has become a trend of current medical treatments. Along with the development of medical imaging equipments, endoscopic surgery has become a representative of minimal invasive surgery. The endoscopic surgery not brings a revolutionary change to medical treatments only, but also reduces the post-operation pain to patients. Even so, the endoscopic surgery still needs to create several minor wounds on a patient's body in order to let an endoscopic device enter into the patient's body for the surgical operation. Therefore, the patient still has to bear with an acute postoperative pain and requires a follow-up treatment of possible wound scars. Obviously, such surgery has an adverse impact on the quality of life of the patients.

However, many imaging systems are used for entering the natural orifices of a patient's body for different medical treatments as the medical imaging equipments have been advanced. With the limitations of surgical devices and technologies, scarless surgical operations through natural orifices are still immature. In recent years, the technology of natural orifice translumenal endoscopic surgery (NOTES) has been developed rapidly, and different surgical operations can be performed with an endoscope passed through a natural orifice (such as an oral cavity, a colon cavity, and a birth channel) then through an internal incision into the body. The postoperative wound is reduced significantly, and a scarless operation can be achieved without affecting the patient's appearance and, thus, NOTES has many advantages over laparoscopy and thoracoscopy. However, NOTES requires more sophisticated surgical skills and only an experienced endoscopic surgery doctor to achieve a high success rate and safety.

The concept of the natural orifice translumenal endoscopic surgery was proposed in 2004. Current literatures related to the successful experience of animal experiments include abdominal and laparoscopic explorations plus liver section surgery, ovary and partial hysterectomy, gastrojejunostomy, tubal ligation, cholecysto-jejunostomy, cholecystectomy, abdominal lymphadenectomy, hernioplasty and proctocolectomy, etc. In 2005, surgical doctors first adopted the NOTES technology on human surgery for appendectomy, and then other NOTES surgeries taken place are laparoscopic exploration and cholecystectomy.

Popular subjects of the NOTES include: how to select an endoscopic entry, prevention of a postoperative infection, sewing-up a postoperative endoscopic entry, and design of endoscopic equipments. Among them, the design and development of the endoscope are the first step of the development of NOTES. Only the best design and user-friendly surgical machinery can provide a good foundation for the surgery and, thus, it is necessary to design appropriate surgical machinery for a specific natural orifice to achieve the effects of NOTES.

In view of the aforementioned shortcomings of the prior art, the inventor of the present invention based on years of experience in the related industry to conduct extensive researches and experiments, and finally developed a NOTES device in accordance with the present invention to overcome the shortcomings of the prior art.

### SUMMARY OF THE INVENTION

It is a primary objective of the present invention to overcome the shortcomings of the prior art by providing a natural orifice translumenal endoscopic surgery (NOTES) device which comprises: a puncture needle, a plurality of dilator sheaths and a plurality of working sheaths. The puncture needle comprises a puncture end, and a protruding safety stud installed at a position substantially at a middle section of the puncture needle; the puncture needle is provided for puncturing into an internal wall of nature lumen, and the nature lumen wall can be a tracheal wall, a vessel wall, or an internal wall of any other natural orifice. The dilator sheaths have a plurality of tapered first diameters, and each dilator sheath has an insert end and a position limit end, and each dilator sheath is sheathed sequentially onto the puncture needle, and the position limit end is stopped at the safety stud of the puncture needle, such that the insert end will not exceed the puncture end and will form a safety interval from the puncture end. The working sheaths have a plurality of tapered second diameters, and each of the second diameters of the working sheaths is slightly greater than each of the first diameters of the dilator sheaths respectively. The working sheath is provided for inserting or retracting the dilator sheaths and the puncture needle after the dilator sheaths have expanded a natural orifice, and the working sheath is remained to form a space provided for the surgical operation.

### BRIEF DESCRIPTION OF THE DRAWINGS

FIG. 1 is a perspective view of a puncture needle in accordance with a preferred embodiment of the present invention;

FIG. 2 is a perspective view of a dilator sheath in accordance with a preferred embodiment of the present invention;

FIG. 3 is a perspective view of a working sheath in accordance with a preferred embodiment of the present invention;

FIG. 4 is a perspective view of a portion of a cutting steel needle in accordance with a preferred embodiment of the present invention;

FIG. 5 is a side view of a puncture needle coupled to a dilator sheath in accordance with a preferred embodiment of the present invention.

### DETAILED DESCRIPTION OF THE PREFERRED EMBODIMENTS

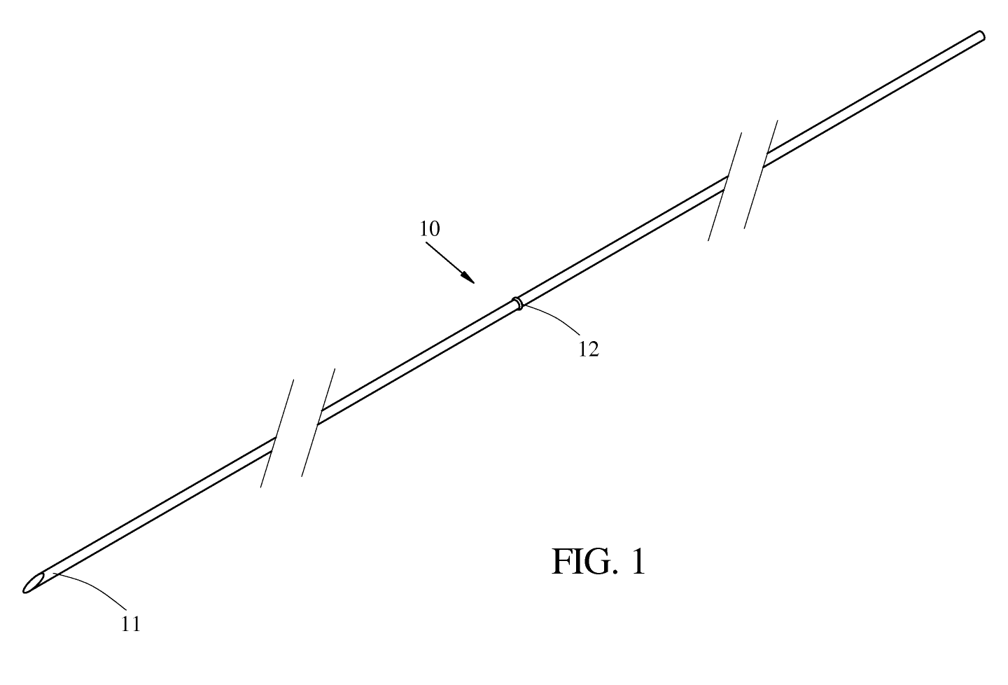

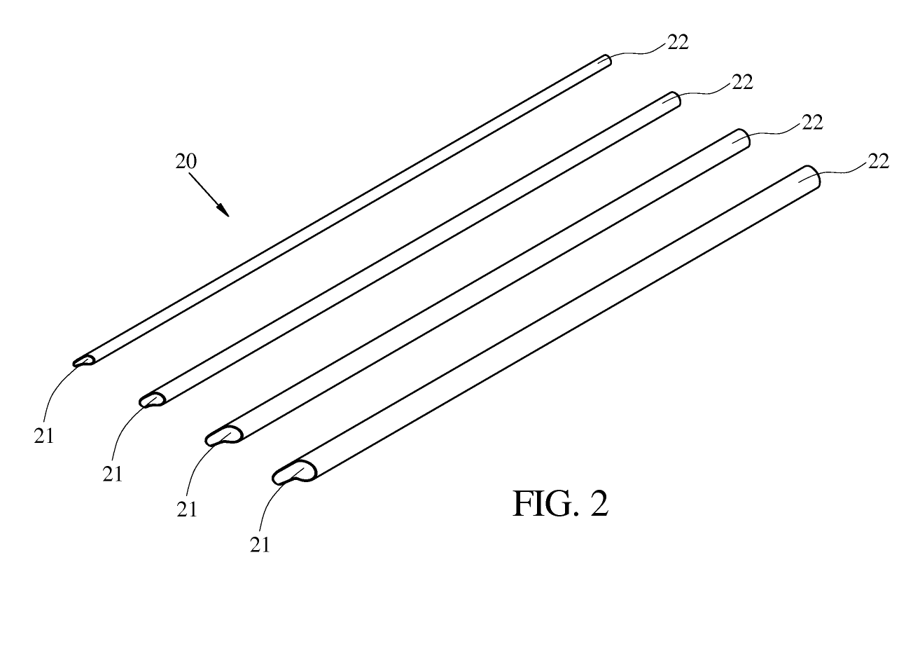

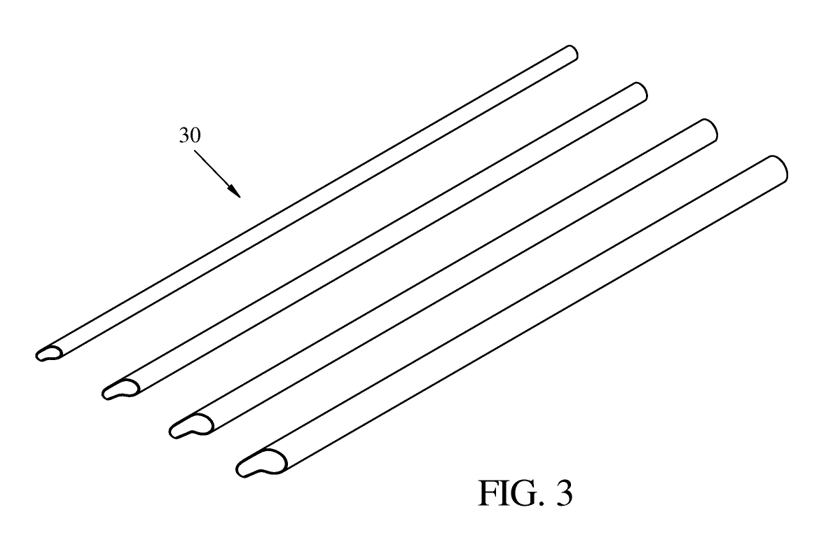

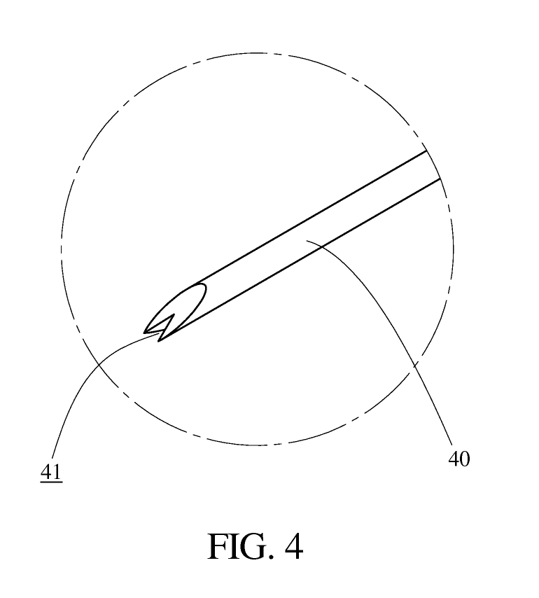

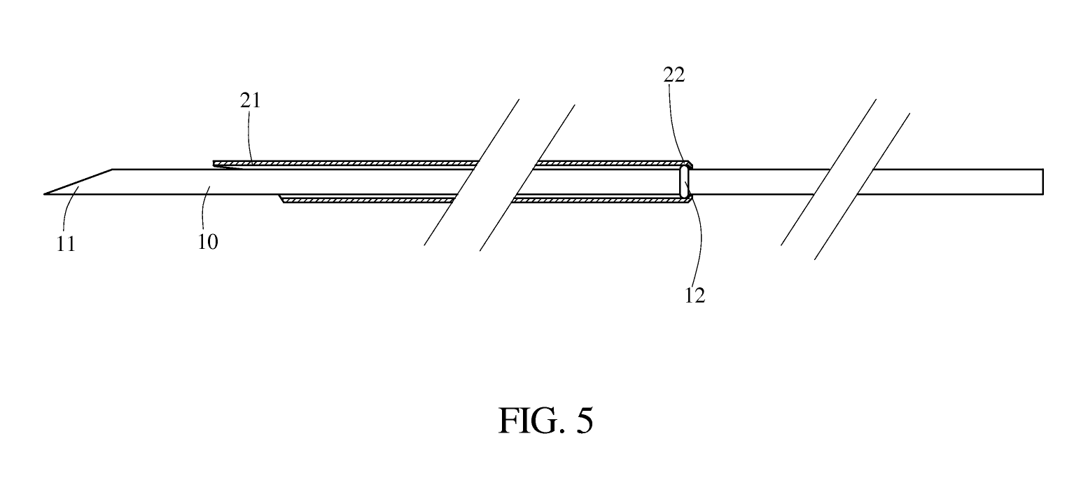

With reference to FIGS. 1 to 5 for a NOTES device of the present invention, the device comprises: a puncture needle 10, a plurality of dilator sheaths 20 and a plurality of working sheaths 30, wherein the puncture needle 10 comprises a puncture end 11, a protruding safety stud 12 installed substantially at a position of a middle section of the puncture needle 10, and the puncture needle 10 is provided for puncturing into an internal wall of nature lumen, wherein the nature lumen wall can be a tracheal wall, a vessel wall, or an internal wall of other natural orifices. In the present invention, an embodiment of the tracheal wall is used for the illustration. The dilator sheaths 20 comprise a plurality of tapered first diameters (or a series of different successively increasing diameters), an insert end 21 and a position limit end 22, and each dilator sheath 20 is sheathed sequentially onto the puncture needle 10, and the position limit end 22 is stopped at the protruding safety stud 12 of the puncture needle 10, such that the insert end 21 will not exceed the puncture end 11 and will form a safety interval from the puncture end. The working sheaths 30 have a plurality of tapered second diameters (or a series of different successively increasing diameters), and each of the second diameters of the working sheaths 30 is slightly greater than each of the first diameters of the dilator sheaths 20, and the working sheath 30 is used for inserting and retracting the dilator sheaths 20 and the puncture needle 10 after the dilator sheaths 20 have expanded a natural orifice, and only the working sheath 30 is remained to form a space required for the surgical operation. In addition, a breathing apparatus can be connceted when the working sheath 30 is used.

The NOTES device further comprises a steel cutting needle 40.

The steel cutting needle 40 has a sharp slanting surface and a groove 41 concavely disposed at the sharp slanting surface for embedding and cutting a surgical thread.

The groove 41 has a substantially V-shaped cross-section.

The puncture needle 10 has a first length substantially equal to 70 cm.

The dilator sheath 20 has a second length substantially equal to 33 cm, and the safety interval is substantially equal to 2 cm.

The plurality of first diameters can be equal to 5 mm, 7 mm, 9 mm and 11 mm respectively; in other words, the dilator sheaths 20 have diameters equal to 5 mm, 7 mm, 9 mm, and 11 mm sequentially for expanding an orifice.

The plurality of second diameters can be equal to 7 mm, 9 mm, 11 mm, and 13 mm respectively; in other words, the plurality of working sheaths 30 can have diameters of 7 mm, 9 mm, 11 mm, and 13 mm respectively for maintaining the smoothness of the orifice during the operation.

In general, the endoscope is entered from a trachea during a surgical operation, and a rigid (or soft) bronchoscopy is used for the surgical operation, and then the puncture needle 10 is inserted into the trachea, and the plurality of dilator sheaths 20 expand an orifice, and a working sheath 30 is used for maintaining the smoothness of the orifice during the surgical operation, and then a rigid bronchoscopy machinery (a rigid one has been used clinically) is adopted for a surgical operation, and the steel cutting needle 40 is used for cutting a surgical thread to complete the surgical operation.

In addition, the method or procedure of the present invention comprises the steps of:

a. Creating a stable airway by a rigid bronchoscopy;

b. Selecting an appropriate puncture position;

c. Passing a surgical sheath from a tracheal sidewall situated at a position substantially 3 cm from the top of a tracheal carina and out of a trachea through a natural orifice;
c1. Passing a puncture needle 10 out of the tracheal sidewall;
c2. Passing a series of dilator sheaths 20 with tapered diameters along the puncture needle 10 sequentially to create an opening;
c3. Preventing unnecessary damages by a design of a safety stud 12 which latches the dilator sheath 20 at a position substantially 2 cm from the tip of the puncture needle 10;
c4. Expanding the opening sequentially, and then retracting the dilator sheath 20 to a position of a sheath with one size smaller than the desired working sheath 30;
c5. Passing the working sheath 30 through the puncture needle 10 and the opening into a pleural space;

d. Using the rigid bronchoscopy machinery for the surgical operation after creating a working channel;
d1. Exchanging air by the working sheath 30 of the rigid bronchoscopy during the surgical operation;
d2. Prohibiting a removal of the sheath during the surgical operation;

e. Making sure that the oxygen level of an experimental animal or a patient reaches 100% before removing the working sheath 30;

f. Removing the working sheath 30 after the air exchange is terminated;

g. Immediately installing a tracheal stent after the working sheath 30 is removed;
g1. Selecting the tracheal stent primarily by using a silicone stent (which can be removed easily), and evaluating the tracheal stent by a 3D CT before the surgical operation takes place;
g2. Covering the opening of the trachea to avoid a pneumothorax;
g3. Performing the endoscopic examination to confirm the position of the stent after the tracheal stent is installed, wherein if the wound is smaller than one-third of the external circumference of the trachea, then the trachea will be healed automatically; and

h. Performing a rigid bronchoscopic examination after the surgical operation takes place, and performing a stent removal operation if the incision healing condition is good. In general, the tracheal stent is covered onto a wound orifice, and the tracheal stent can be removed after two weeks when the wound has been recovered naturally.

In summary, the present invention provides a NOTES device that passes through a trachea to achieve the advantages of the NOTES, and the design of the device provides a user-friendly and easy operation for a scarless surgical operation without affecting a patient's appearance. In addition, the present invention further includes a protruding safety stud installed at a position substantially at a middle section of the puncture needle, such that the dilator sheath can be latched at a position substantially 2 cm from the tip of the puncture needle to prevent unnecessary damages.

What is claimed is:

1. A natural orifice translumenal endoscopic surgery (NOTES) device, comprising:
a puncture needle, including a puncture end and a protruding safety stud installed at a position substantially at a middle section of the puncture needle, and the puncture needle being provided for puncturing into an internal wall of a nature lumen;
a plurality of dilator sheaths, having a plurality of tapered first diameters, an insert end and a position limit end, and each dilator sheath being sheathed sequentially onto the puncture needle, and the position limit end being stopped at the protruding safety stud of the puncture needle, such that the insert end does not exceed the puncture end and forms a safety interval from the puncture end; and
a plurality of working sheaths, having a plurality of tapered second diameters, and each of the tapered second diameters of the working sheaths being slightly greater than the tapered first diameters of the dilator sheaths respectively, and the working sheath being provided for inserting and retracting the dilator sheaths and the puncture needle to form a space required for a surgical operation after the dilator sheaths have expanded a natural orifice.

2. The NOTES device of claim 1, further comprising a steel cutting needle.

3. The NOTES device of claim 2, wherein the steel cutting needle has a sharp slanting surface, and a groove concavely disposed on the sharp slanting surface for embedding and cutting a surgical thread.

4. The NOTES device of claim 3, wherein the groove has a substantially V-shaped cross-section.
